(19) United States
(12) Patent Application Publication
(10) Pub. No.: US 2007/0173703 A1
(43) Pub. Date: Jul. 26, 2007

(54) Method, apparatus, and medium for managing weight by using calorie consumption information

(75) Inventors: Kwang Hyeon Lee, Yongin-si (KR); Hye Jin Jung, Seoul (KR); Sang Hoon Shin, Seongnam-si (KR); Kyung Ho Kim, Yongin-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/475,053

(22) Filed: Jun. 27, 2006

(30) Foreign Application Priority Data
Dec. 6, 2005 (KR) 10-2005-118020

(51) Int. Cl. A61B 5/00

(52) U.S. Cl. 600/300; 128/921; 128/905

(57) ABSTRACT

A method of managing weight by using calorie consumption information, the method including: receiving target information from a user; receiving calorie consumption data of the user; analyzing the calorie consumption data based on the target information; and providing feedback to the user for a result of the analyzing the calorie consumption data.

### CROSS-REFERENCE TO RELATED APPLICATIONS

This application claims the benefit of Korean Patent Application No. 10-2005-118020, filed on Dec. 6, 2005, in the Korean Intellectual Property Office, the disclosure of which is incorporated herein by reference.

### BACKGROUND OF THE INVENTION

1. Field of the Invention

The present invention relates to a method, apparatus, and medium for managing weight by using calorie consumption information of a user, and more particularly, to a method, apparatus, and medium for managing weight, which provide detailed information with respect to exercise intensity or exercise pattern of a user by analyzing calorie consumption information of the user.

2. Description of the Related Art

Currently, it is a trend that the overweight population continuously increases due to changes in environment and nutrition. Complications due to obesity and/or being overweight emerge as a greater problem than obesity and/or the state of being overweight itself.

Now, since information with respect to how many calories an obese and/or overweight patient intakes and consumes depends on a diet diary and an exercise diary written by the obese and/or overweight patient in weight clinics, there is no method of providing feedback on everyday life of the obese and/or patient, thereby generating a problem of not effectively providing weight management services. In addition, individuals who are not overweight and wish to maintain their weight also desire feedback on the activities of their everyday life.

Korean Patent Laid-Open Publication No. 2002-0050316 discloses a conventional wireless communication device equipped with a pedometer function and a health management method and system using the device, in which a burden of directly inputting information on food intake is on a user, and a simple exercise amount of the user may be known. Also, in the conventional health management method, it is advised to consume all intake calories by exercising but there is a problem of not being able to compute calorie consumption of walking and ordinary tasks in addition to an inputted exercise amount.

Korean Patent Laid-Open Publication No. 2003-0010828 discloses a conventional Internet walking meter system in which an activity graph, a heartbeat, blood sugar value, and calorie intake may be exchanged by universal serial bus (USB) communication via data stored in an Internet walking meter and a function of measuring a number of steps and a function of data transmission terminal may be performed. However, since the conventional Internet walking meter system checks an activity amount by using the number of steps of a user and weight loss is planned by using the checked activity amount without recognition with respect to calories taken by the user, a precise weight loss plan is difficult to create.

Japanese Patent Laid-Open No. 2002-263086 discloses a method of providing an activity condition and a degree of consumed calories by using acceleration and angular velocity data. However, since a result is simply shown only in a graph, a method of determining and analyzing a calorie consumption pattern may not be provided.

In Japanese Patent Laid-Open No. 2004-227522, consumed calories are computed based on an exercise time and progress of past consumed calories is shown in a graph. However, only activity and energy consumption pattern of a user may be viewed is disclosed and there is no analysis method on how to examine in detail.

In Japanese Patent Laid-Open No. 2000-116630, data on walking is measured for an extended period and shown and a key for measuring data with respect to walking is included by using a mobile phone, and the measured data is provided to a user as feedback. However, there is a limitation in that a basic level of feedback is provided by using only information with respect to consumed calories.

Japanese Patent Laid-Open Publication 1996-131425 discloses an activity amount measurement device capable of recognizing consumed calories for each activity. However, the device only computes consumed calories for each activity by using an acceleration sensor and a method of analyzing exercise pattern of a user is not disclosed.

As described above, since the conventional activity amount measurement device executes a function as an auxiliary means, the device may be difficult to be used for systematic and continuous weight management.

Also, since the conventional activity amount measurement device transmits measured activity amount data to a server in order to analyze the measured activity amount data, a user cannot directly receive a result of an analysis of the measured activity amount data.

In addition, in the conventional activity amount device, since information provided as feedback to the user is just an amount of calories consumed by exercise, detailed data analysis result desired by the user may not be provided.

Accordingly, instead of simply measuring and providing an activity amount or calorie consumption amount of the user, a method of providing feedback to the user on an activity pattern acquired by analyzing the measured activity amount or calorie consumption amount is greatly desired.

### SUMMARY OF THE INVENTION

Additional aspects, features, and/or advantages of the invention will be set forth in part in the description which follows and, in part, will be apparent from the description, or may be learned by practice of the invention.

An aspect of the present invention provides a method, apparatus, and medium for managing weight, which may provide feedback to a user on a detailed result of analyzing calorie consumption information of the user.

Also, according to the present invention, whether a user aerobically exercises more than a certain amount of time in a day may be easily recognized according to the result of analyzing the calorie consumption information of the user.

An aspect of the present invention also provides a method, apparatus, and medium for managing weight, which may provide feedback to a user on an activity time pattern of the user by analyzing a calorie consumption time slot.

An aspect of the present invention also provides a method, apparatus, and medium for managing weight, which may provide feedback to a user on an exercise intensity rate with respect to a degree of intensity the user exercises for a day by analyzing one day calorie consumption information of the user.

An aspect of the present invention also provides a method, apparatus, and medium for managing weight, which may provide feedback to a user on whether the user regularly exercises by analyzing calorie consumption information of the user.

An aspect of the present invention also provides a method, apparatus, and medium for managing weight, which may provide feedback to a user on activity pattern information with respect to whether the user has a weekend exercise pattern or a weekday exercise pattern, by analyzing one week calorie consumption information of the user.

An aspect of the present invention also provides a method, apparatus, and medium for managing weight, which may provide feedback to a user by an alarm for requesting activity at set intervals in real time, by analyzing calorie consumption information and weight information of the user.

An aspect of the present invention also provides a method, apparatus, and medium for managing weight, which may provide feedback to a user, in real time, of a point in time of lipolysis in a case of sustaining aerobic exercise, by analyzing calorie consumption information and weight information of the user.

An aspect of the present invention also provides a method, apparatus, and medium for managing weight, which may provide feedback to a user on calorie information of foods ingested by the user by analyzing calorie consumption information and weight information of the user.

According to an aspect of the present invention, there is provided a method of managing weight by using calorie consumption information, the method including: receiving target information from a user; receiving calorie consumption data of the user; analyzing the calorie consumption data based on the target information; and providing feedback to the user of a result of the analyzing the calorie consumption data.

According to another aspect of the present invention, there is provided a method of managing weight by using calorie consumption information, the method including: receiving activity information from a user; receiving calorie consumption data of the user; analyzing amount of activity of the user by using the inputted calorie consumption data; and providing feedback to the user for a predetermined activity request information according to the analyzed activity amount of the user in real time.

According to another aspect of the present invention, there is provided a method of managing weight by using calorie consumption information, the method including: obtaining and processing weight data and calorie consumption data of a user; determining a calorie consumption standard according to an amount of a weight change with respect to a standard intake of the user; and estimating caloric intake of the user according to the weight change and the calorie consumption of the user based on the determined standard.

According to another aspect of the present invention, there is provided at least one computer readable medium storing instructions that control at least one processor to perform a method of managing weight by using calorie consumption information, the method including: receiving target information from a user; receiving calorie consumption data of the user; analyzing the calorie consumption data based on the target information; and providing feedback to the user of a result of the analyzing the calorie consumption data

According to still another aspect of the present invention, there is provided an apparatus for managing weight by using calorie consumption information, the apparatus including: an input unit for receiving target information inputted from a user; a receiving unit receiving calorie consumption data and weight data of the user; an analysis unit analyzing the calorie consumption data based on the target information and the weight data; and a feedback unit providing feedback to the user of a result of analyzing the calorie consumption data.

According to yet another aspect of the present invention, there is provided an apparatus for managing weight by using calorie consumption information, the apparatus including: an input unit for receiving target information inputted from a user; a weight information receiving unit receiving weight information of the user; a calorie consumption measurement unit measuring calorie consumption of the user; an analysis unit analyzing exercise amount of the user based on the target information, the weight information, and the calorie consumption; and a feedback unit providing feedback to the user of the analyzed exercise amount and a prescription corresponding to the analyzed exercise amount.

According to another aspect of the present invention, there is provided at least one computer readable medium storing instructions that control at least one processor to perform a method of managing weight by using calorie consumption information, the method including: receiving activity information from a user; receiving calorie consumption data of the user; analyzing an amount of activity of the user by using the inputted calorie consumption data; and providing feedback to the user for a predetermined activity request information according to the analyzed activity amount of the user in real time.

According to another aspect of the present invention, there is provided at least one computer readable medium storing instructions that control at least one processor to perform a method of managing weight by using calorie consumption information, the method including: obtaining and processing weight data and calorie consumption data of a user; determining a calorie consumption standard according to an amount of a weight change with respect to a standard intake of the user; and estimating caloric intake of the user according to the weight change and the calorie consumption of the user based on the determined standard.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. These and/or other aspects, features, and advantages of the invention will become apparent and more readily appreciated from the following description of exemplary embodiments, taken in conjunction with the accompanying drawings of which:

FIG. 1 is a configuration diagram illustrating an apparatus for managing weight by using calorie consumption information, according to an exemplary embodiment of the present invention;

FIG. 2 is a configuration diagram illustrating an apparatus for managing weight by using calorie consumption information, according to another exemplary embodiment of the present invention;

FIG. 3 is a diagram illustrating an example of calorie consumption amount for each time slot with respect to a user, in the apparatus for managing weight;

FIG. 4 is a flowchart illustrating a method of managing weight by using calorie consumption information, according to an exemplary embodiment of the present invention;

FIG. 5 is a flowchart illustrating an example of a procedure for determining a number of times of aerobically exercising for longer than a certain amount of time in one day and for a greater intensity than a certain intensity, in the method of managing weight according to an exemplary embodiment;

FIG. 6 is a flowchart illustrating an example of a procedure for determining an activity pattern of the user, in the method of managing weight according to an exemplary embodiment;

FIG. 7 is a flowchart illustrating an example of a procedure for determining an exercise intensity ratio of the user, in the method of managing weight according to an exemplary embodiment;

FIG. 8 is a flowchart illustrating an example of a procedure for determining whether the user aerobically exercises regularly, in the method of managing weight according to an exemplary embodiment;

FIG. 9 is a flowchart illustrating an example of a procedure for determining whether the user is a weekend activity type or a weekday activity type, in the method of managing weight according to an exemplary embodiment;

FIG. 10 is a flowchart illustrating an example of a procedure for determining whether the user exercises irregularly, in the method of managing weight according to an exemplary embodiment;

FIG. 11 is a flowchart illustrating a method of managing weight by using calorie consumption information, according to another exemplary embodiment of the present invention;

FIG. 12 is a diagram illustrating an example of estimating intake calories, in the method of managing weight according to an exemplary embodiment;

FIG. 13 is a flowchart illustrating an example of a procedure for providing an activity alarm at a certain time, in the method of managing weight according to an exemplary embodiment;

FIG. 14 is a flowchart illustrating an example of a procedure for notifying a lipolysis point in time during continued exercise in the method of managing weight according to an exemplary embodiment;

FIG. 15 is a flowchart illustrating an example of a procedure for estimating food intake calories, in the method of managing weight according to an exemplary embodiment; and

FIG. 16 is a diagram illustrating an example of food intake calories according to calorie consumption amount and weight change.

### DETAILED DESCRIPTION OF THE PREFERRED EMBODIMENTS

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

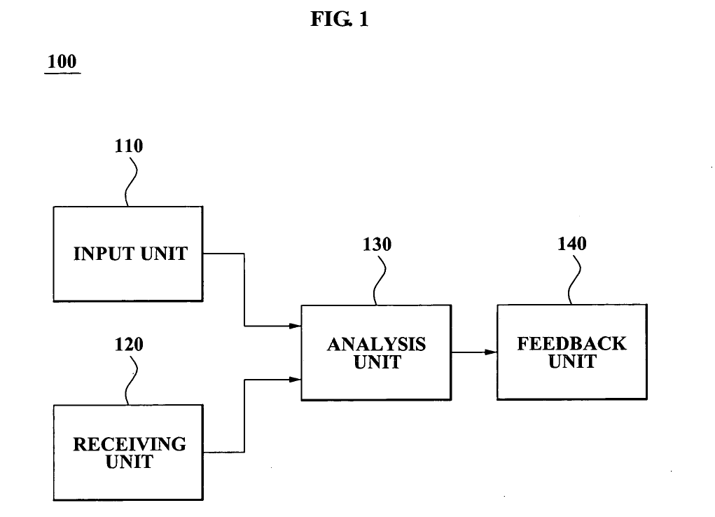

FIG. 1 is a configuration diagram illustrating an apparatus 100 for managing weight by using calorie consumption information, according to an exemplary embodiment of the present invention.

Referring to FIG. 1, the apparatus 100 includes an input unit 110, a receiving unit 120, an analysis unit 130, and a feedback unit 140. The apparatus 100 may be embodied within a portable device such as a Personal Digital Assistant (PDA), a smart phone, a handheld PC, and a mobile phone. The apparatus 100 may analyze calorie consumption information of a user and provide feedback of a result of analyzing to the user. The feedback may be a message indicating past activity of a user and/or suggesting future activity to the user. The feedback may also be an alarm, beep, or other signal indicating past activity or suggesting an activity to a user.

The input unit 110 receives target information from the user. The target information may include a target exercise duration, a target exercise intensity, a target exercise intensity for each activity, or a target number of days of exercising.

The receiving unit 120 receives calorie consumption data and weight data of the user. The receiving unit 120 may receive calorie consumption data measured by a predetermined calorie tracker. Also, the receiving unit 120 may receive weight data of the user, measured by a predetermined weight measurement device. The weight measurement device may be, for example, shoes capable of measuring weight.

The analysis unit 130 analyzes the calorie consumption data based on the target information and the weight data. The analysis unit 130 may determine whether the user aerobically exercises for longer than a certain time and for a greater intensity than a certain intensity for one day, by analyzing the calorie consumption data based on the target information and the weight data. The analysis unit 130 may determine the user to be a morning activity type, an afternoon activity type, an evening activity type, or a midnight activity type, by analyzing time information and the calorie consumption data. The analysis unit 130 may determine a ratio of exercise by the user, the exercises have different intensities, such as light, moderate, and strenuous, by analyzing the target information and the calorie consumption data. The analysis unit 130 may determine whether the user regularly or irregularly exercises, by analyzing the target information and the calorie consumption data. The analysis unit 130 may determine whether the user is a weekend activity type or a weekday activity type, by analyzing the target information and the week's calorie consumption data of the user. The analysis unit 130 may recognize an activity request point in time of the user by analyzing the target information and the calorie consumption data. The analysis unit 130 may recognize a lipolysis point in time of the user by analyzing the target information and the calorie consumption data.

The feedback unit 140 provides feedback of a result of analyzing the calorie consumption data to the user in real time. The feedback unit 140 may provide a message notifying whether the user exercises longer than a certain time and more than a certain intensity for one day, to the user, as the result of analyzing the calorie consumption data. The feedback unit 140 may provide an exercise time pattern with respect to whether the user is a morning activity type, an afternoon activity type, an evening activity type, or a midnight activity type, to the user, as the result of analyzing one day calorie consumption data of the user. The feedback unit 140 may provide a ratio of exercises by the user, which have different intensity such as light, moderate, or strenuous exercises, to the user as the result of analyzing the calorie consumption data of the user. The feedback unit 140 may provide a message notifying whether the user regularly or irregularly exercises, to the user, as the result of analyzing the calorie consumption data. The feedback unit 140 may provide an exercise time pattern with respect to whether the user is a weekend activity type or a weekday activity type, to the user, as the result of analyzing one week calorie consumption data of the user. The feedback unit 140 may provide an alarm for requesting activity of the user, to the user, as the result of analyzing the calorie consumption data. The feedback unit 140 may provide an alarm for notifying a lipolysis point in time of the user, to the user, as the result of analyzing the calorie consumption data. The feedback unit 140 may provide feedback of the result of analyzing the calorie consumption data to a predetermined user health management server. When the apparatus 100 is a portable device or embodied within a portable device such as a mobile phone, PDA, etc., the result of analyzing the calorie consumption data may be transmitted to the user health management server via a base station. Also, the apparatus 100 may transmit the result of analyzing the calorie consumption data to the user health management server via a PC.

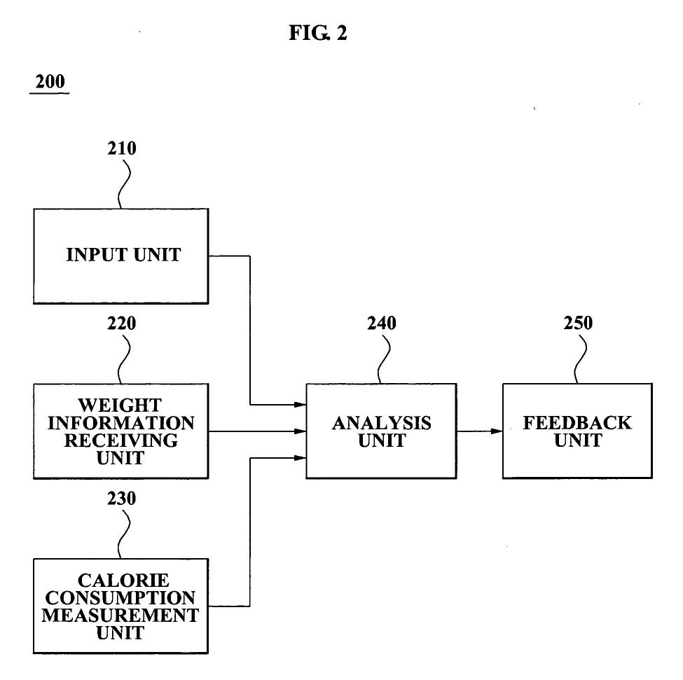

FIG. 2 is a configuration diagram illustrating an apparatus 200 for managing weight by using calorie consumption information, according to another exemplary embodiment of the present invention.

Referring to FIG. 2, the apparatus 200 includes an input unit 210, a weight information receiving unit 220, a calorie consumption measurement unit 230, an analysis unit 240, and a feedback unit 250. The feedback may be a message indicating past activity of a user and/or suggesting future activity to the user. The feedback may also be an alarm, beep, or other signal indicating past activity or suggesting an activity to a user.

The apparatus 200 may be embodied within a portable device such as a PDA, a smart phone, a handheld PC, and a mobile phone. The apparatus 200 may analyze calorie consumption information and provide a result of analyzing the calorie consumption information to the user in real time.

The input unit 210 receives target information from the user. The target information may include a target exercise duration, a target exercise intensity, a target exercise intensity for each activity, or a target number of days of exercising.

The weight information receiving unit 220 receives weight data of the user. Also, the weight information receiving unit 220 may receive weight information of the user, measured by a predetermined weight measurement device. The weight measurement device may be, for example, shoes capable of measuring weight.

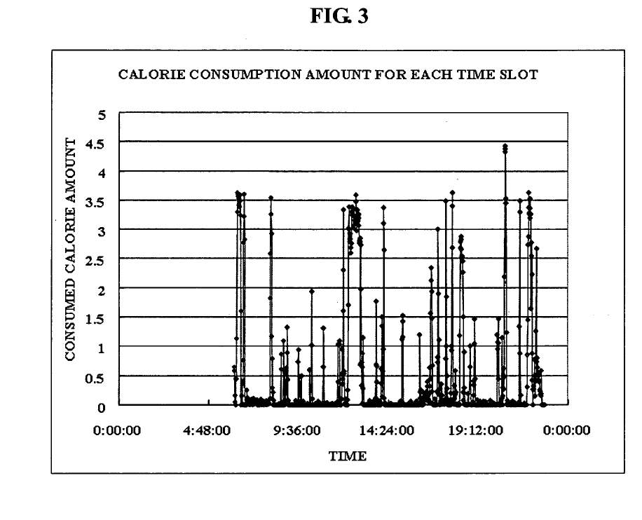

The calorie consumption measurement unit 230 measures an amount of calories consumed for each time slot with respect to the user as shown in FIG. 3. The amount of consumed calories provides calorie consumption data.

The analysis unit 240 analyzes an exercise amount of the user based on the target information, the weight information, and the calorie consumption amount. The analysis unit 240 may determine whether the user aerobically exercises for longer than a certain time and more than a certain intensity for one day, based on the target information, the weight information, and the calorie consumption amount. The analysis unit 240 may determine the user to be a morning activity type, an afternoon activity type, an evening activity type, or a midnight activity type, based on exercise time information and the calorie consumption amount of the user. The analysis 240 may determine a ratio of exercises by the user, the exercises having different intensity, such as light, moderate, and strenuous, based on the target information and the calorie consumption amount. The analysis unit 240 may determine whether the user regularly or irregularly exercises, based on the target information and the calorie consumption amount. The analysis 240 may determine whether the user is a weekend activity type or a weekday activity type, based on the target information and the week calorie consumption amount of the user. The analysis unit 240 may analyze an activity request point in time of the user based on the target information and the calorie consumption amount. The analysis unit 240 may analyze a lipolysis point in time of the user based on the target information and the calorie consumption amount.

The feedback unit 250 provides the analyzed exercise amount and a prescription corresponding to the analyzed exercise amount to the user, as feedback.

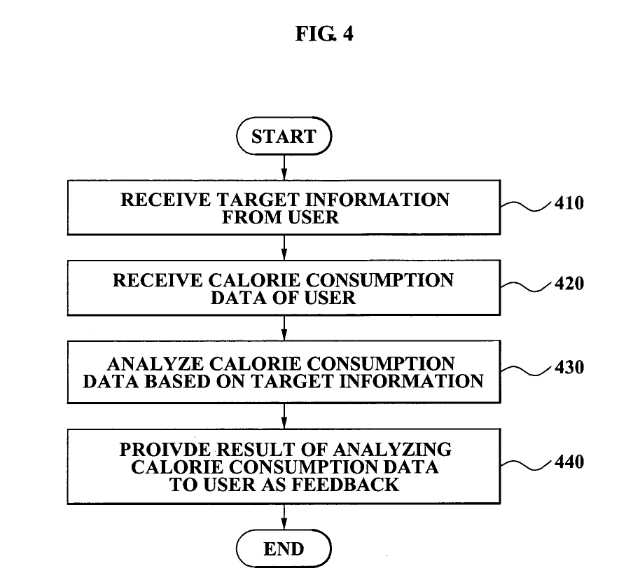

FIG. 4 is a flowchart illustrating a method of managing weight by using calorie consumption information, according to an exemplary embodiment of the present invention.

Referring to FIG. 4, in operation 410, an apparatus for managing weight receives target information from a predetermined user. The target information may include a target exercise duration, a target exercise intensity, a target exercise intensity for each activity, or a target number of days of exercising.

In operation 420, the apparatus receives calorie consumption data from the user. The apparatus may receive calorie consumption data of the user, according to time, from a predetermined calorie measurement unit. Namely, the apparatus may receive a calorie consumption amount for each time slot of the user, as shown in FIG. 3, from the calorie measurement unit.

In operation 430, the apparatus analyzes the calorie consumption data based on target information. The apparatus may analyze an amount of consumed calories exceeding a predetermined standard value for a duration designated according to the target information by using the inputted calorie consumption data. The apparatus may analyze a time slot in which most calories are consumed by using one day calorie consumption data of the user. The apparatus may analyze exercise intensity of the user by using the calorie consumption data. The apparatus may analyze an exercise pattern of the user for a week, with respect to whether the user is a weekend exercise pattern or a weekday exercise pattern, by using week's calorie consumption data of the user. The apparatus may determine the exercise pattern of the user to be irregular by using the week's calorie consumption data, when more than a determined ratio of calories to total consumed calories of the user is consumed within three days.

In operation 440, the apparatus provides a result of analyzing the calorie consumption data to the user as feedback. The apparatus may provide the analyzed number as a number of times that the user aerobically exercises at an intensity greater than a certain intensity determined according to the target information. The apparatus may provide feedback to the user on whether the exercise pattern of the user is a weekday exercise type or a weekend exercise type.

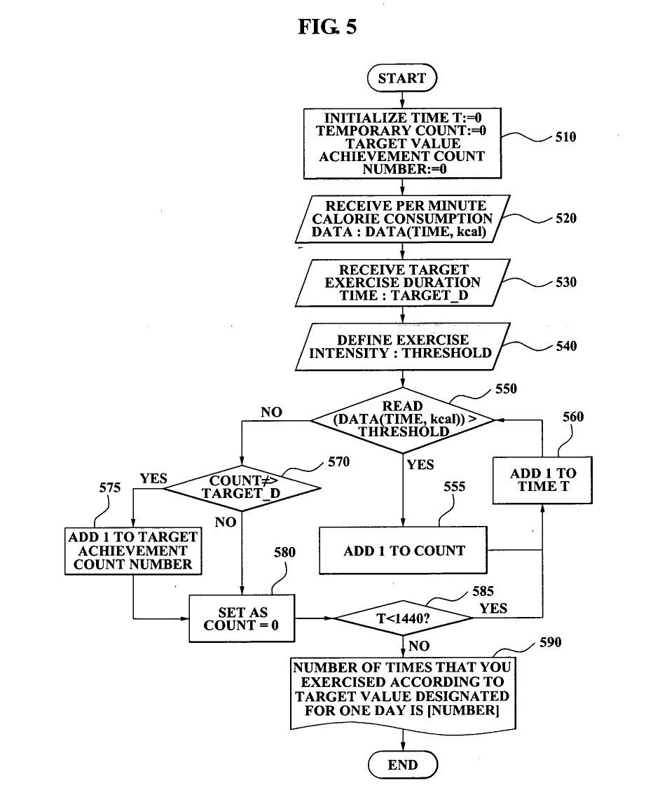

FIG. 5 is a flowchart illustrating an example of a procedure for determining a number of times of aerobically exercising for longer than a certain amount of time in one day and for a greater intensity than a certain intensity, in the method of managing weight according to an exemplary embodiment.

Referring to FIG. 5, in operation 510, the apparatus initializes time, a temporary count, and a count of a number of target value achievements.

In operation 520, the apparatus receives per minute calorie consumption data for one day of the user from a predetermined calorie tracker. The per minute calorie consumption data for one day of the user may include time information in which calories are consumed and consumed calorie information. When a pedometer is used instead of the calorie tracker, the apparatus may receive step number data instead of the calorie data.

In operation 530, the apparatus may receive target exercise duration time information Target_D from the user.

In operation 540, the apparatus determines a threshold of exercise intensity. The threshold of the exercise intensity may be, for example, 5 kcal in the case of running, 2.5 kcal in the case of quick walking, and 1 kcal in the case of slow walking.

In operation 550, the apparatus reads the inputted calorie consumption data and determines whether an amount of consumed calories is more than the threshold.

When the calorie consumption data is more than the threshold, the apparatus adds 1 to the count in operation 555.

In operation 560, the apparatus adds 1 to the time. This is for scanning the calorie consumption data from one minute to 1440 minutes (one full day) because the calorie consumption data is inputted each minute.

When the calorie consumption data is not more than the threshold, the apparatus determines whether the count is more than the target exercise duration time Target_D in operation 570. Namely, the apparatus may determine whether the time in which more calories are consumed than the threshold is for longer than the target exercise duration time.

When the count is more than the target exercise duration time, the apparatus adds 1 to the target achievement count number in operation 575.

When the count is not more than the target exercise duration time or after adding 1 to the target achievement count number, the apparatus sets the count as 0 in operation 580.

In operation 585, the apparatus determines whether the time is less than 1440.

When the time is less than 1440, the apparatus performs the operations starting from operation 560.

When the time is not less than 1440, the apparatus provides a message as “A number of times that you exercised according to a target value designated for one day is [number].” to the user as feedback in operation 590. This message is an example of a message provided as feedback to the user.

As described above, in the method of managing weight according to an exemplary embodiment, the calorie consumption data is analyzed and the number of times that the user aerobically exercises according to the target of an exercise prescription of the user for one day is provided to the user as feedback.

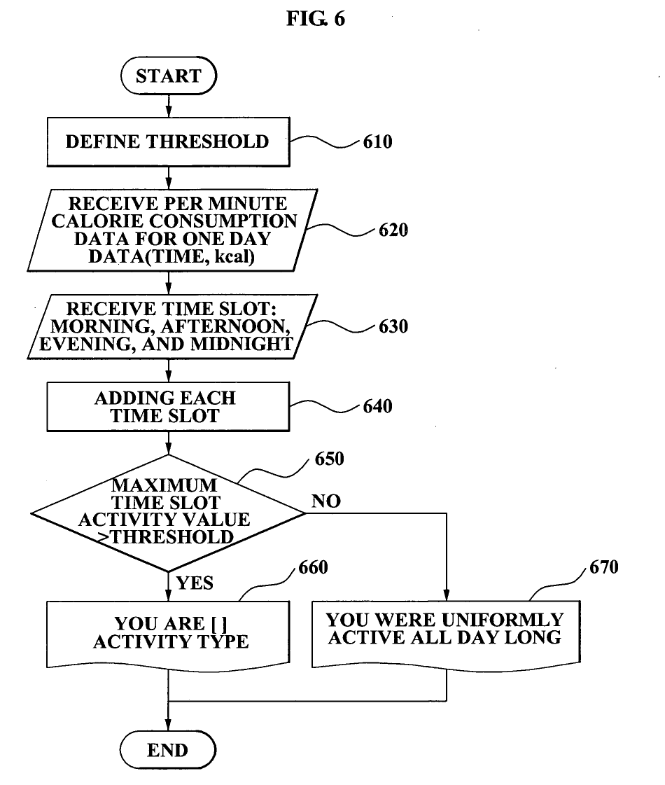

FIG. 6 is a flowchart illustrating an example of a procedure for determining an activity pattern of the user, in the method of managing weight according to an exemplary embodiment.

Referring to FIG. 6, in operation 610, the apparatus defines a threshold for determining an activity pattern of the user.

In operation 620, the apparatus receives per minute calorie consumption data for one day.

In operation 630, the apparatus receives time slot information such as morning, afternoon, evening, or midnight. The time slot information may include time information, for example, 6 to 8 o'clock in the case of morning, 11 to 13 o'clock in the case of afternoon, 17 to 19 o'clock in the case of evening, and 23 to 1 o'clock in the case of midnight.

In operation 640, the apparatus adds up calorie consumption data for each time slot.

In operation 650, the apparatus determines whether the added calorie consumption data of a maximum time slot, whose added calorie consumption data value is greater than other time slots, is greater than the threshold.

When the added calorie consumption data value of the maximum time slot is more than the threshold, in operation 660, the apparatus provides time slot information in which the user is usually active, to the user as feedback, by using a message, for example, “You are [ ] activity type.”.

When the maximum time slot is in the morning and the added calorie consumption data of the morning is more than the threshold, in operation 660, the apparatus may provide a message as, for example, “You are a morning activity type. Please, be sure to have breakfast because you are usually active in the morning.”, to the user.

When the maximum time slot is in the afternoon and the added calorie consumption data of the afternoon is more than the threshold, in operation 660, the apparatus may provide a message as, for example, “You are an afternoon activity type. Please, exercise after having lunch and have a snack before supper because you may feel hungry.”, to the user.

When the maximum time slot is evening and the added calorie consumption data of the evening is more than the threshold, in operation 660, the apparatus may provide a message as, for example, “You are an evening activity type. Please, eat plenty at supper and refrain from a midnight or heavy snack even if you are hungry after exercising.”, to the user.

When the added calorie consumption data of the maximum time slot is less than the threshold, in operation 670, the apparatus provides feedback to the user as the user was uniformly active all day long instead of being usually active in an established time slot, by using a message as “You were uniformly active all day long.”.

As described above, in the method of managing weight, according to an exemplary embodiment, the one day calorie consumption data of the user is analyzed and the activity time pattern in a time slot the user is usually active is determined to provide feedback of the information of the activity time pattern to the user. The messages provided to the user are examples of messages provided to the user as feedback.

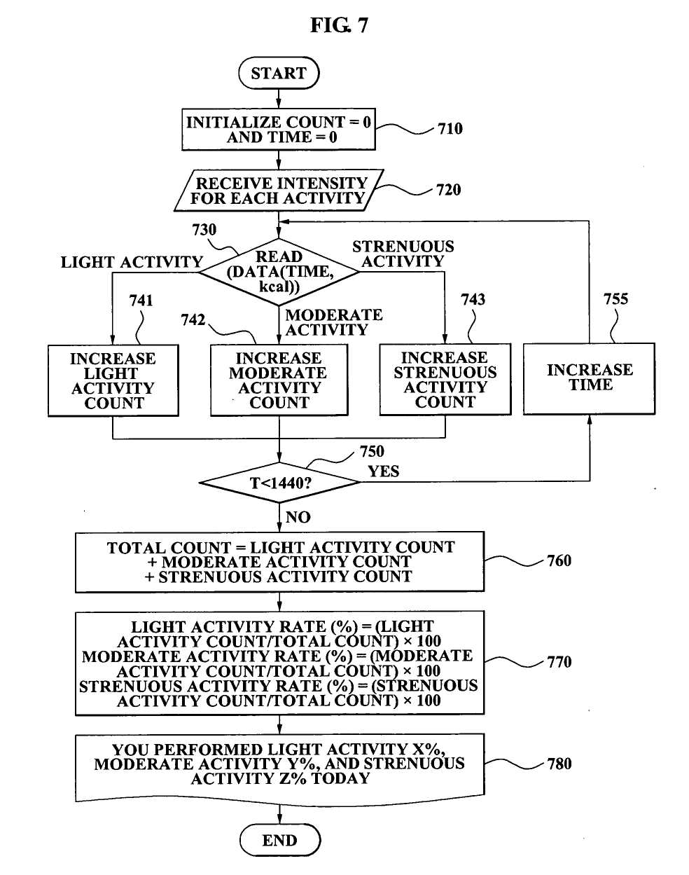

FIG. 7 is a flowchart illustrating an example of a procedure for determining an exercise intensity ratio of the user, in the method of managing weight according to an exemplary embodiment.

Referring to FIG. 7, in operation 710, the apparatus initializes variables such as count and time. The count may include light activity count, moderate activity count, or strenuous activity count.

In operation 720, the apparatus receives intensity for each activity. The intensity for each activity may have different exercise intensity information such as light activity, moderate activity, or strenuous activity. For example, intensity of the light activity may correspond to the calorie consumption data of 0 to 1 kcal, intensity of the moderate activity may correspond to the calorie consumption data of 1 to 4 kcal, and intensity of the strenuous activity may correspond to the calorie consumption data of more than 4 kcal.

In operation 730, the apparatus reads calorie consumption data inputted according to each time and determines an intensity the calorie consumption data corresponds to, the light activity, moderate activity, or strenuous activity.

When the calorie consumption data corresponds to the light activity intensity, the apparatus increases a count of the light activity in operation 741.

When the calorie consumption data corresponds to the moderate activity intensity, the apparatus increases a count of the moderate activity in operation 742.

When the calorie consumption data corresponds to the strenuous activity intensity, the apparatus increases a count of the strenuous activity in operation 743.

In operation 750, the apparatus determines whether the time T of the calorie consumption data is less than 1440.

When the time of the calorie consumption data is less than 1440, the apparatus increases the time in operation 755.

When the time of the calorie consumption data is not less than 1440, the apparatus computes a total count value by adding a light activity count value, a moderate activity count value, and a strenuous activity count value. In other words, when the time of the calorie consumption data is not less than 1440, the calorie consumption data for all day long is summed in operation 760.

In operation 770, the apparatus computes a light activity rate X, a moderate activity rate Y, and a strenuous activity rate Z, respectively. The light activity rate X is “light activity count value/a total count value×100”, the moderate activity rate Y is “moderate activity count value/the total count value×100”, and the strenuous activity rate Z is “strenuous activity count value/the total count value×100”.

In operation 780, the apparatus provides feedback to the user as a message of, for example, “You perform light activity X %, moderate activity Y %, and strenuous activity Z % today.”.

As described above, in the method of managing weight, according to an exemplary embodiment, one day calorie consumption data of the user is analyzed and the rate of exercise intensity, indicating that the user exercises a certain amount for each of the intensities for one day is analyzed, thereby providing feedback to the user.

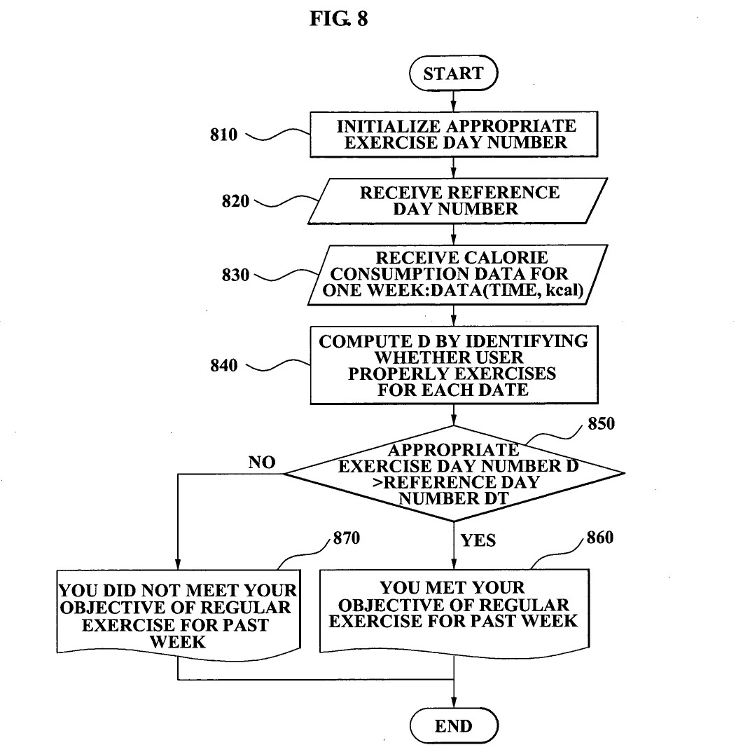

FIG. 8 is a flowchart illustrating an example of a procedure for determining whether the user aerobically exercises regularly, in the method of managing weight according to an exemplary embodiment.

Referring to FIG. 8, in operation 810, the apparatus initializes a variable such as an appropriate exercise day number D.

In operation 820, the apparatus receives a reference day number (DT). The reference day number DT is a reference for identifying a number of days the user regularly exercises for a week. For example, when it is identified that the user regularly exercises 5 days for a week, the reference day number DT becomes “5”.

In operation 830, the apparatus receives per minute calorie consumption data for a week.

In operation 840, the apparatus computes the proper exercise day number D by identifying whether the user properly exercises for each date by analyzing the received per minute calorie consumption data for a week. Namely, the apparatus determines that the user has properly exercised when the per minute calorie consumption data for each date is analyzed and more calories than a predetermined standard value are consumed, and may also compute dates of properly exercising.

In operation 850, the apparatus determines whether the computed proper exercise day number D is more than the reference day number DT.

When the appropriate exercise day number D is greater than the reference day number DT, in operation 860, the apparatus provides feedback to the user as a message indicating that the user aerobically exercise regularly for a determined period as, for example, “You met your objective of regular exercise for the past week.”.

When the appropriate exercise day number D is not more than the reference day number DT, in operation 870, the apparatus provides feedback to the user as a message indicating that regular aerobic exercise is not taken for a determined period as, for example, “You did not meet your objective of regular exercise for the past week.”.

As described above, in the method of managing weight, according to an exemplary embodiment, whether the user regularly exercises for the determined period is determined by analyzing the calorie consumption data and a message corresponding to the determination may be provided to the user as feedback.

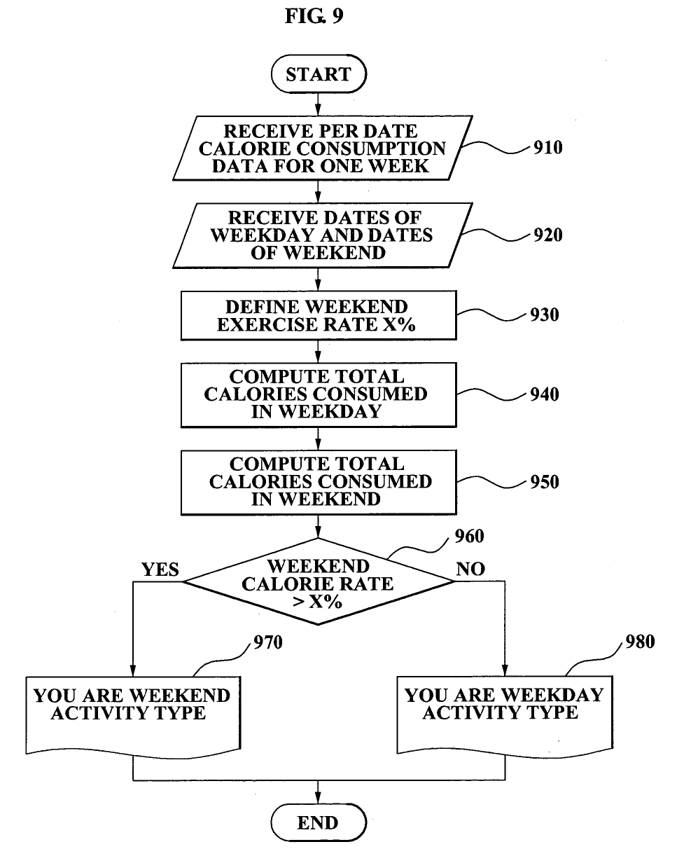

FIG. 9 is a flowchart illustrating an example of a procedure for determining whether the user is a weekend activity type or a weekday activity type, in the method of managing weight according to an exemplary embodiment.

Referring to FIG. 9, in operation 910, the apparatus receives per date calorie consumption data of one week. Also, when the apparatus itself measures calorie consumption data of the user, the apparatus may store the measured calorie consumption data of the user for each date and may read the per date calorie consumption data of one week of the user.

In operation 920, the apparatus receives dates of weekdays and dates of weekend. Namely, the apparatus may receive, for example, the dates of October 4 to 7 for the weekdays and the dates of October 8 and 9 for the weekend. Also, when the apparatus includes a function of a calendar or clock, the operation of receiving the dates of the weekdays or the dates of the weekend may be omitted.

In operation 930, the apparatus defines a rate of X % for weekend exercise. The rate X % for weekend exercise may be used as a reference for determining whether the user is a weekday activity type or a weekend activity type.

In operation 940, the apparatus computes total calories consumed during the weekdays by analyzing the received per date calorie consumption data of one week.

In operation 950, the apparatus computes total calories consumed during the weekend and a weekend calorie rate by analyzing the received per date calorie consumption data of one week. The weekend calorie rate is computed by “(sum of calories consumed in weekend/sum of total consumed calories)×100”.

In operation 960, the apparatus determines whether the weekend calorie rate is more than the defined weekend exercise rate X %.

When the weekend calorie rate is more than the defined weekend exercise rate X %, in operation 970, the apparatus provides feedback to the user as a message indicating that the user has a pattern of exercising during the weekend as, for example, “You are a weekend activity type.”.

Also, in operation 970, when the user is a weekend activity type, the apparatus may provide further feedback to the user as a message of advice as, for example “You should exercise in smaller amounts. If you do all your exercise during the weekend, you may easily feel fatigued and may not be consistently counseled.”.

When the weekend calorie rate is not more than the defined weekend exercise rate X %, in operation 980, the apparatus may provide feedback to the user as a message indicating that the user has a pattern of exercising during the weekdays as, for example, “You are a weekday activity type.”.

Also, in operation 980, when the user has the weekday activity type pattern, the apparatus may provide further feedback to the user as a message as, for example, “Daily fatigue may be increased due to exercise along with weekday activity. Increase caloric intake and shift some exercises to weekends.”.

As described above, in the method of managing weight, according to an exemplary embodiment, the user's calorie consumption data of one week is analyzed to determine whether the user is the weekend activity type or the weekday activity type and a result of the determination may be provided to the user as feedback.

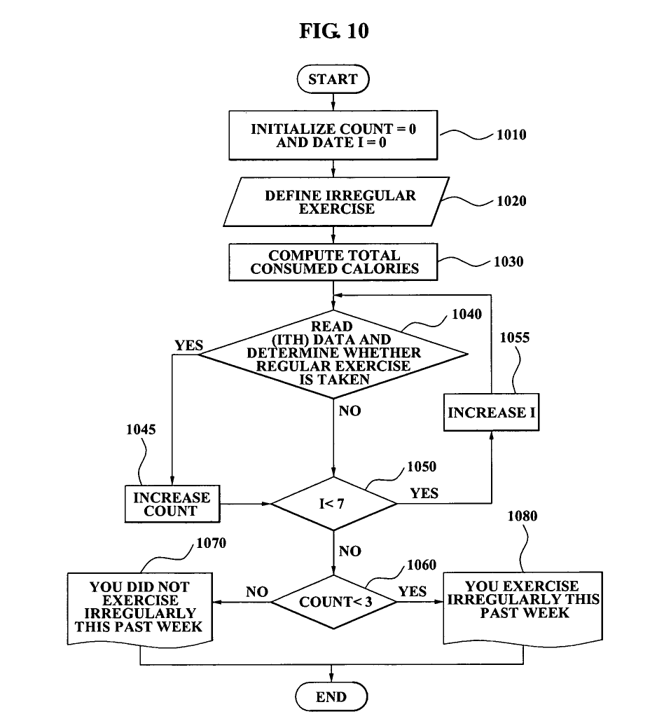

Referring to FIG. 10, in operation 1010, the apparatus initializes variables such as a count and a date i.

In operation 1020, the apparatus defines irregular exercise. For example, the apparatus may define a case of consuming more than 30% of total consumed calories in one or two days, as the irregular exercise.

In operation 1030, the apparatus receives per date calorie consumption data of one week and computes total consumption calories with respect to the inputted calorie consumption data.

In operation 1040, the apparatus reads calorie consumption data of ith date and determines whether regular exercise is taken on the ith date by analyzing the calorie consumption data of the ith date.

When the regular exercise is taken on the ith date, the apparatus increases the count in operation 1045.

When the regular exercise is not taken on the ith date or the count is increased, the apparatus determines whether the number of days i is less than 7 in operation 1050.

When the i is less than 7, the apparatus increases the i in operation 1055 and again performs the operation of 1040.

When the i is not less than 7, the apparatus determines whether the count is less than 3 in operation 1060.

When the count is not less than 3, the apparatus may provide feedback to the user as a message notifying that the user did not exercise irregularly as, for example, “You did not exercise irregularly this past week.” in operation 1070.

When the count is less than 3, the apparatus may provide feedback to the user as a message notifying that the user exercises irregularly as, for example, “You exercised irregularly this past week.” in operation 1080.

As described above, in the method of managing weight, according to an exemplary embodiment, whether the user exercises irregularly for one week may be determined by analyzing the calorie consumption data of one week and a result of the determination may be provided to the user as feedback.

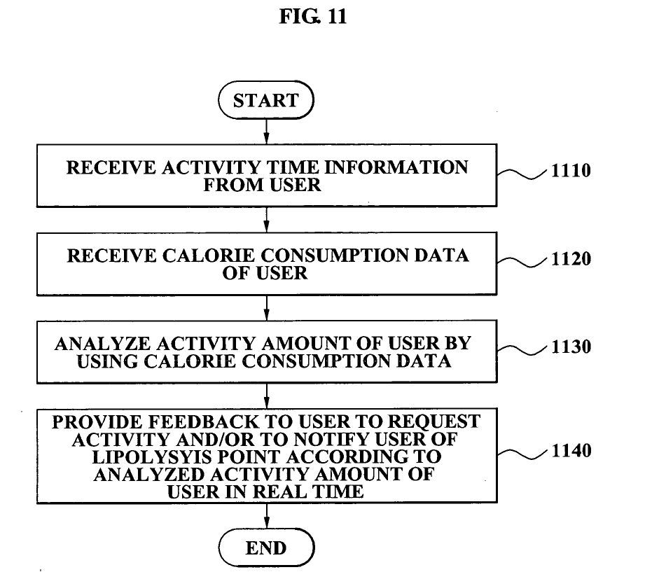

FIG. 11 is a flowchart illustrating a method of managing weight by using calorie consumption information, according to another exemplary embodiment of the present invention.

Referring to FIG. 11, the apparatus receives activity information from the user. The activity information may include activity period, a threshold for exercise intensity with respect to a rest/idleness, and activity time information in operation 1110.

In operation 1120, the apparatus receives the calorie consumption data of the user. Also, in operation 1120, when including a calorie measurement unit capable of measuring the calorie consumption data of the user, the apparatus may measure the calorie consumption data of the user by using the calorie measurement unit.

In operation 1130, the apparatus analyzes an activity amount of the user by using the inputted calorie consumption data. The apparatus may monitor weight data of the user in real time and may compute calories taken in by the user based on the calorie data and weight data. The apparatus considers the weight data of the user to be valid weight data only when the weight data of the user is within 80 to 120% of an original weight data. Namely, when the weight data of the user is out of the 80 to 120% range of the original weight data, the apparatus may determine the weight data of the user to be incorrectly measured.

When the weight data of the user does not exist, the apparatus may compute the weight data via linear interpolation using previously measured weight data of the user.

The apparatus may compute weight change data by comparing first weight data measured at a first point in time with second weight data measured at a second point in time and may analyze calories taken in by the user based on the calorie consumption data and the computed weight change data.

When the periodically monitored weight data of the user is not consecutive, the apparatus may compute weight data between the first point in time and the second point in time via the linear interpolation based on the first weight data measured at the first point in time and the second weight data measured at the second point in time.

The apparatus may analyze the weight change data of the user by comparing the first weight data with the second weight data or by using a gradient of the weight data of the user.

The apparatus may record food ingestion and defecation time data of the user and may adjust the computed weight data by referring to the recorded time data.

When a sudden weight change occurs while the activity amount is regularly maintained, the apparatus does not use the weight data changed due to the food ingestion or defecation of the user in the process of computing the weight data via the linear interpolation.

For example, when there is no activity of the user for more than five minutes and the weight of the user increases more than 400 g during that period, the apparatus may determine that the user has ingested a meal. For example, when the weight of the user suddenly increases by approximately 100 to 400 g regardless of the activity amount of the user, the apparatus may determine that the user has ingested snacks or beverages.

For example, when the activity amount of the user does not exist for more than three minutes and the weight of the user is reduced by more than 400 g, the apparatus may determine that the user has a movement. For example, when the weight of the user is reduced by approximately 100 to 400 g, the apparatus may determine that the user has urinated.

In operation 1130, when an amount of weight change data is less than the calorie consumption data, the apparatus may determine intake calories of the user as high-calorie. Also, when the weight change amount is more than the calorie consumption amount of the user, the apparatus may determine the user ingests low-caloric food.

In operation 1140, when analyzed activity amount of the user is less than an activity amount established according to the activity information, the apparatus may provide feedback to the user by an alarm for requesting activity, in real time.

Also, in operation 1140, the apparatus may record a point in time when the calorie consumption is maintained for longer than a predetermined standard value, according to the analyzed activity amount of the user and may provide feedback to the user by an alarm for notifying that the recorded point in time when more time passes than a predetermined time is a lipolysis point in time, in real time.

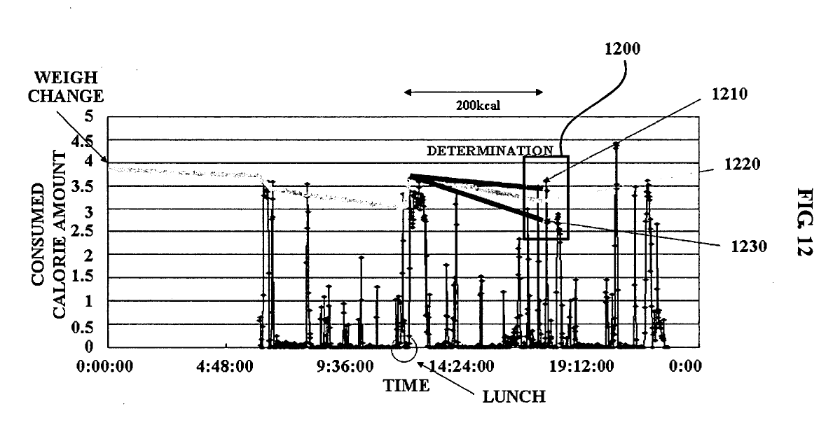

FIG. 12 is a diagram illustrating an example of estimating intake calories, in the method of managing weight according to an exemplary embodiment.

Referring to FIG. 12, the apparatus analyzes the user's calorie consumption amount and weight change data for each time slot and estimates the user's intake calories from a meal at a point in time 1200 when a certain time passes after the user has the meal and the calorie consumption amount increases and the weight is reduced.

First data 1210 is a case in which a decrease of the weight of the user is less than the calorie consumption amount of the user and is determined as the user has ingested high-caloric foods.

Second data 1220 is a case in which the calorie consumption amount and the decrease of the weight of the user are suitable and is determined as the user has ingested foods having appropriate calories.

Third data 1230 is a case in which the decrease of the weight of the user is more than the calorie consumption amount of the user and is determined as the user has ingested low-caloric foods.

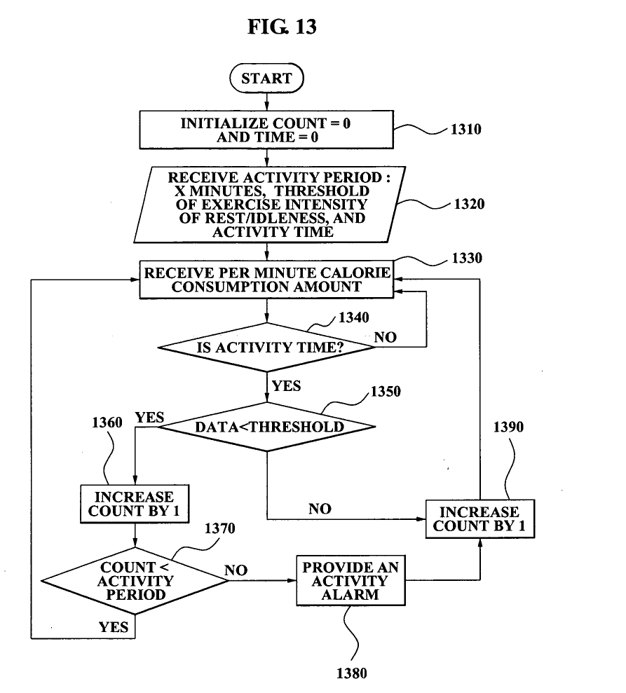

FIG. 13 is a flowchart illustrating an example of a procedure for providing an activity alarm at a certain time, in the method of managing weight according to an exemplary embodiment.

Referring to FIG. 13, in operation 1310, the apparatus initializes variables associated with the count and the time.

In operation 1320, the apparatus receives an activity period, a threshold of exercise intensity with respect to a rest/idleness, and an activity time. The activity period may be inputted per minute.

In operation 1330, the apparatus receives per minute calorie consumption amount from a predetermined calorie tracker. When the calorie consumption amount of the user is measured by itself, the apparatus may measure the calorie consumption amount of the user every minute.

In operation 1340, the apparatus determines whether it is the received activity time. When it is not the activity time, the apparatus performs the operation of 1330.

In operation 1350, the apparatus determines whether the calorie consumption amount data is less than the threshold of the exercise intensity.

When the calorie consumption amount data is less than the threshold of the exercise intensity, the apparatus increases a count by 1 in operation 1360.

In operation 1370, the apparatus determines whether the count is less than the activity period.

When the count is not less than the activity period, the apparatus provides an alarm for requesting the user to perform an activity in operation 1380.

When the calorie consumption amount data is not less than the threshold of the exercise intensity or after the alarm for requesting activity is provided, the apparatus increases the count by 1 in operation 1390 and again performs the operation of 1330.

As described above, in the method of managing weight, according to an exemplary embodiment, the per minute calorie consumption amount of the user is analyzed and may provide the alarm for requesting the user to perform activity when the time in which the user does not exercise more than the received threshold of exercise intensity in the activity period is longer than the received activity period.

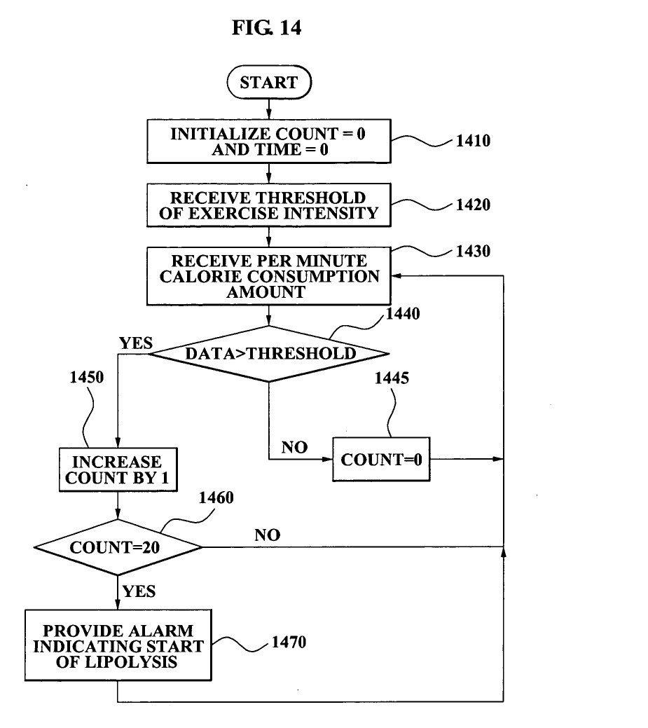

FIG. 14 is a flowchart illustrating an example of a procedure for notifying a lipolysis point in time in continued exercise in the method of managing weight according to exemplary embodiment.

Referring to FIG. 14, the apparatus initializes variables such as a count and a time in operation 1410.

In operation 1420, the apparatus receives a threshold of exercise intensity, at which lipolysis may start.

In operation 1430, the apparatus receives per minute calorie consumption amount from a calorie tracker. When capable of measuring calorie consumption amount of the user by itself, the apparatus may measure the calorie consumption amount of the user every minute.

In operation 1440, the apparatus determines whether the calorie consumption amount Data is more than the threshold of the exercise intensity.

When the calorie consumption amount Data of the user is not more than the threshold, the apparatus initializes the count as “0” in operation 1445 and again performs the operation of 1430.

When the calorie consumption amount of the user is more than the threshold, the apparatus increases the count by 1 in operation 1450.

In operation 1460, the apparatus determines whether the count is “20”. The count is “20” is for sensing that a point in time that a time of taking exercise more than a certain threshold becomes “20” by analyzing the calorie consumption amount of the user.

When the count is “20”, the apparatus provides an alarm notifying that lipolysis starts, to the user in operation 1470.

As described above, in the method of managing weight, according to an exemplary embodiment, the calorie consumption amount of the user is analyzed, the time of exercising is counted according to the analyzed calorie consumption amount, and the alarm notifying that the lipolysis starts at the point in time that continued exercise time becomes 20 minutes is provided to the user, thereby exercising more in order to reduce body fat of the user.

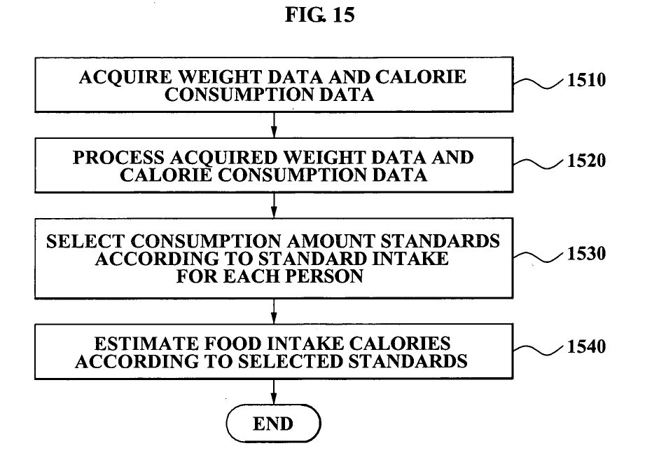

FIG. 15 is a flowchart illustrating an example of a procedure for estimating food intake calories, in the method of managing weight according to exemplary embodiment.

Referring to FIG. 15, the apparatus acquires weight data and calorie consumption data of the user in operation 1510. Namely, the apparatus acquires the weight data from a weight measurement device of the user and acquires the calorie consumption data from a calorie tracker of the user.

In operation 1520, the apparatus processes the acquired weight data and the calorie consumption data. Namely, when there are too much weight data and an interval therebetween is less than certain standards, the apparatus may adjust the weight data by using linear interpolation. Also, when the acquired weight data are insufficient and the interval between the weight data is more than the certain standards, the apparatus may adjust the interval between the weight data by referring to the calorie consumption data of the user. Also, when there is a point in which a weight of the user suddenly decreases/increases while an activity amount of the user is regularly maintained, since that point is not due to a weight change according to actual calorie consumption but an effect due to defecation or snacks, the point may be excluded from adjusting the weight data by using the linear interpolation.

In operation 1530, the apparatus selects consumption amount standards in according to personal standard intake of the user. Namely, the apparatus may induce the user to have a meal according to determined calories and weight, may collect a calorie consumption amount and weight change data of the user, may record times of defecation and snack intake to adjust the weight change data, and may select calorie consumption amount standards, personal average, in proportion to a weight change amount according to personal standard intake amount of the user.

On the other hand, the apparatus may provide calorie information according to a type and weight of foods to the user in order to induce the user to have a meal according to the determined calories and weight in operation 1530.

In operation 1540, the apparatus estimates food intake calories according to the meal of the user based on the selected standards. In this case, the apparatus may estimate calories of foods taken by the user between each meal, such as a breakfast and a lunch, by checking the meal of the user.

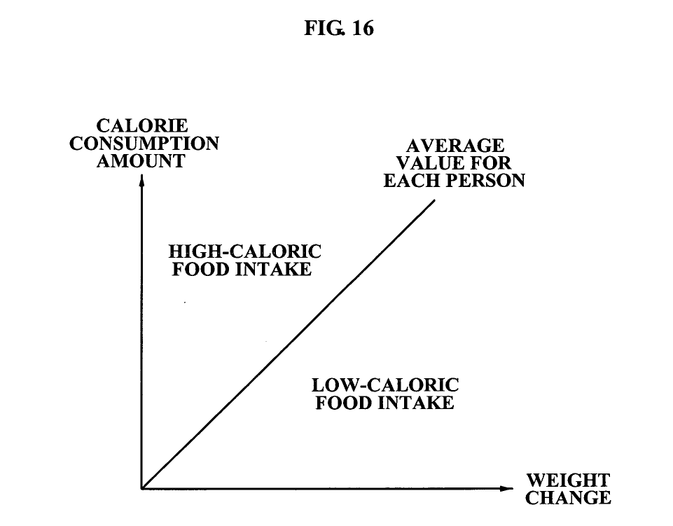

When the weight change is determined to be less than the calorie consumption amount of the user based on the selected standards, as shown in FIG. 16, the apparatus may determine that the user ingested high-caloric food.

When the weight change is determined to be more than the calorie consumption amount of the user based on the selected standards, as shown in FIG. 16, the apparatus may determine that the user ingested low-caloric food.

As described above, in the method of managing weight, according to an exemplary embodiment, the calories of the food taken by the user may be estimated according to the weight change and calorie consumption amount of the user and may be provided as food intake information.

According to the present invention, there are provided a method and apparatus for managing weight, which may provide feedback to a user on a detailed result of analyzing calorie consumption information of the user.

Also, according to the present invention, whether a user aerobically exercises more than a certain amount of time in a day may be easily recognized according to the result of analyzing the calorie consumption information of the user.

Also, according to the present invention, there are provided a method and apparatus for managing weight, which may provide feedback to a user on an activity time pattern of the user by analyzing a calorie consumption time slot.

Also, according to the present invention, there are provided a method and apparatus for managing weight, which may provide feedback to a user on an exercise intensity rate with respect to a degree of intensity the user exercises for a day by analyzing one day calorie consumption information of the user.

Also, according to the present invention, there are provided a method and apparatus for managing weight, which may provide feedback to a user on whether the user regularly exercises by analyzing calorie consumption information of the user.

Also, according to the present invention, there are provided a method and apparatus for managing weight, which may provide feedback to a user on activity pattern information with respect to whether the user has a weekend exercise pattern or a weekday exercise pattern, by analyzing one week calorie consumption information of the user.

Also, according to the present invention, there are provided a method and apparatus for managing weight, which may provide feedback to a user by an alarm for requesting activity at set intervals in real time, by analyzing calorie consumption information and weight information of the user.

Also, according to the present invention, there are provided a method and apparatus for managing weight, which may provide feedback to a user, in real time, of a point in time of lipolysis in a case of sustaining taking aerobic exercise, by analyzing calorie consumption information and weight information of the user.

Also, according to the present invention, there are provided a method and apparatus for managing weight, which may provide feedback to a user on calorie information of foods ingested by the user by analyzing calorie consumption information and weight information of the user.

Also, according to the present invention, there are provided a method and apparatus for managing weight, which may provide feedback to a user. The feedback may be a message indicating past activity of a user and/or suggesting future activity to the user. The feedback may also be an alarm, beep, or other signal indicating past activity or suggesting an activity to a user

In addition to the above-described exemplary embodiments, exemplary embodiments of the present invention can also be implemented by executing computer readable code/instructions in/on a medium, e.g., a computer readable medium. The medium can correspond to any medium/media permitting the storing and/or transmission of the computer readable code.

The computer readable code/instructions can be recorded/transferred in/on a medium in a variety of ways, with examples of the medium including magnetic storage media (e.g., floppy disks, hard disks, magnetic tapes, etc.), optical recording media (e.g., CD-ROMs, or DVDs), magneto-optical media (e.g., floptical disks), hardware storage devices (e.g., read only memory media, random access memory media, flash memories, etc.) and storage/transmission media such as carrier waves transmitting signals, which may include instructions, data structures, etc. Examples of storage/transmission media may include wired and/or wireless transmission (such as transmission through the Internet). Examples of wired storage/transmission media may include optical wires and metallic wires. The medium/media may also be a distributed network, so that the computer readable code/instructions is stored/transferred and executed in a distributed fashion. The computer readable code/instructions may be executed by one or more processors.

Although a few exemplary embodiments of the present invention have been shown and described, it would be appreciated by those skilled in the art that changes may be made in these exemplary embodiments without departing from the principles and spirit of the invention, the scope of which is defined in the claims and their equivalents.

1. A method of managing weight by using calorie consumption information, the method comprising:
receiving target information from a user;
receiving calorie consumption data of the user;
analyzing the calorie consumption data based on the target information; and
providing feedback to the user of a result of the analyzing the calorie consumption data.

2. The method of claim 1, wherein, in the receiving calorie consumption data of the user, the calorie consumption data of the user, based on time, is received from a predetermined calorie measurement device.

3. The method of claim 1, wherein, in the analyzing the calorie consumption data, an amount of calories consumed exceeding a predetermined standard value for a duration designated according to the target information is analyzed by using the inputted calorie consumption data.

4. The method of claim 3, wherein the providing feedback to the user for the result of the analyzing the calorie consumption data comprises the operation of providing feedback of the analyzed number as a number that the user aerobically exercises at an intensity greater than an intensity determined according to the target information.

5. The method of claim 1, wherein, in the analyzing calorie consumption data, a time slot of a greatest amount of calories consumed is analyzed by using calorie consumption data for one day, of the user.

6. The method of claim 5, wherein, in the providing feedback to the user for a result of the analyzing the calorie consumption data, an activity time pattern of the user and behavior pattern information corresponding to the activity time pattern of the user, which are obtained by using the analyzed time period information, are provided to the user as the feedback.

7. The method of claim 5, wherein the time slot includes any one of morning, afternoon, evening, or midnight.

8. The method of claim 1, wherein, in the analyzing the calorie consumption data, exercise intensity is analyzed by using the calorie consumption data.

9. The method of claim 8, wherein, in the providing feedback to the user for a result of the analyzing the calorie consumption data, a ratio of the analyzed exercise intensity is provided to the user as the feedback.

10. The method of claim 1, wherein, in the analyzing the calorie consumption data, a week's exercise pattern of the user is analyzed by using a week's calorie consumption data of the user.

11. The method of claim 10, wherein, in the providing feedback to the user for a result of the analyzing the calorie consumption data, according to the analyzed week's exercise pattern of the user, whether the exercise pattern of the user is a weekday-type or weekend-type is provided to the user as the feedback.

12. The method of claim 1, wherein, in the analyzing the calorie consumption data, when more calories than a predetermined ratio of total consumed calories are consumed within three days, the exercise pattern of the user is determined to be irregular by using the week's calorie consumption data.

13. A method of managing weight by using calorie consumption information, the method comprising:
receiving activity information from a user;
receiving calorie consumption data of the user;
analyzing an amount of activity of the user by using the inputted calorie consumption data; and
providing feedback to the user for a predetermined activity request information according to the analyzed activity amount of the user in real time.

14. The method of claim 13, wherein, in the providing feedback to the user for a predetermined activity request information according to the analyzed activity amount of the user in real time, an alarm of requesting the user to act is provided to the user as the feedback in real time when the analyzed activity amount is smaller than a set up activity amount.

15. The method of claim 13, wherein, the providing feedback to the user for a predetermined activity request information according to the analyzed activity amount of the user in real time comprises:
counting a time in which the calorie consumption data is maintained to be more than a predetermined standard value according to the analyzed activity amount; and
providing feedback to the user for an alarm which indicates a point in time that the counted time passes a predetermined time, as a point in time of decomposing body fat, in real time.

16. The method of claim 13, further comprising:
monitoring weight data of the user; and
analyzing caloric intake based on the calorie consumption data and the weight data.

17. The method of claim 16, wherein the analyzing caloric intake based on the calorie consumption data and the weight data comprises:
analyzing weight change data of the user by comparing first weight data measured at a first point in time and second weight data measured at a second point in time; and
analyzing the caloric intake of the user based on the calorie consumption data and the analyzed weight change data.

18. The method of claim 16, further comprising the operation of computing weight data between the first point in time and the second point in time via linear interpolation based on the first weight data measured at the first point in time and the second weight data measured at the second point in time when the monitored weight data of the user is not continuous.

19. The method of claim 17, wherein, in the analyzing weight change data of the user by comparing first weight data measured at a first point in time and second weight data measured at a second point in time, the weight change data of the user is analyzed by comparing the first weight data with the second weight data or by using a gradient of the weight data of the user.

20. The method of claim 18, further comprising:
recording food intake time data of the user and defecation time data of the user; and
adjusting the computed weight data by referring to the recoded time data.

21. The method of claim 17, wherein, in the analyzing the caloric intake of the user based on the calorie consumption data and the analyzed weight change data, the calorie intake of the user is determined to be high caloric when an amount of a change of the weight change data is smaller than the calorie consumption data.

22. A method of managing weight by using calorie consumption information, the method comprising:
obtaining and processing weight data and calorie consumption data of a user;
determining a calorie consumption standard according to an amount of a weight change with respect to a standard intake of the user; and
estimating caloric intake of the user according to the weight change and the calorie consumption of the user based on the determined standard.

23. The method of claim 22, wherein:
the obtaining and processing weight data and calorie consumption data of a user comprises the operation of revising the weight data by using a linear interpolation when an interval of the obtained weight data is less than a certain standard; and
in the revising the weight data, a case of a sudden reduction of the weight of the user when an activity amount of the user is constantly maintained is not included in an objective of the revision according to the linear interpolation.

24. The method of claim 23, wherein the determining a calorie consumption standard according to an amount of a weight change with respect to a standard intake of the user comprises:
collecting the calorie consumption and the weight change data of the user ingesting a meal according to determined calorie and weight;
revising the weight change data by recording defecation time and snack intake time; and
determining the calorie consumption standard according to the weight change with respect to the standard intake of the user based on the calorie consumption and the revised weight change data.

25. The method of claim 22, wherein, in the estimating caloric intake of the user, the caloric intake of the user in a section between meals is estimated by checking the meals of the user.

26. At least one computer readable medium storing instructions that control at least one processor to perform a method of managing weight by using calorie consumption information, the method comprising:
receiving target information from a user;
receiving calorie consumption data of the user;
analyzing the calorie consumption data based on the target information; and
providing feedback to the user of a result of the analyzing the calorie consumption data.

27. An apparatus for managing weight by using calorie consumption information, the apparatus comprising:
an input unit receiving target information inputted from a user;
a receiving unit receiving calorie consumption data and weight data of the user;
an analysis unit analyzing the calorie consumption data based on the target information and the weight data; and
a feedback unit providing feedback to the user of a result of analyzing the calorie consumption data.

28. The apparatus of claim 27, wherein the feedback unit provides the feedback to a predetermined user health management server of the result of analyzing the calorie consumption data.

29. An apparatus for managing weight by using calorie consumption information, the apparatus comprising:
an input unit receiving target information inputted from a user;
a weight information receiving unit receiving weight information of the user;
a calorie consumption measurement unit measuring calorie consumption of the user;
an analysis unit analyzing exercise amount of the user based on the target information, the weight information, and the calorie consumption; and
a feedback unit providing feedback to the user of the analyzed exercise amount and a prescription corresponding to the analyzed exercise amount.

30. At least one computer readable medium storing instructions that control at least one processor to perform a method of managing weight by using calorie consumption information, the method comprising:
receiving activity information from a user;
receiving calorie consumption data of the user;
analyzing an amount of activity of the user by using the inputted calorie consumption data; and
providing feedback to the user for a predetermined activity request information according to the analyzed activity amount of the user in real time.

31. At least one computer readable medium storing instructions that control at least one processor to perform a method of managing weight by using calorie consumption information, the method comprising:
obtaining and processing weight data and calorie consumption data of a user;
determining a calorie consumption standard according to an amount of a weight change with respect to a standard intake of the user; and
estimating caloric intake of the user according to the weight change and the calorie consumption of the user based on the determined standard.
